SEQUENCE LISTING (72 sequences)

SEQ ID NO: 1 - DNA, 26 bp, Artificial Sequence
  the sequence is synthesized
  modified_base (6)..(8): “n” refers to deoxyinosine
gaggtnnnccacacagaactaacctc

SEQ ID NO: 2 - DNA, 26 bp, Artificial Sequence
  the sequence is synthesized
  modified_base (6)..(8): “n” refers to deoxyinosine
ggtttnnnttatgagaaaagcaaacc

SEQ ID NO: 3 - DNA, 24 bp, Artificial Sequence
  the sequence is synthesized
  modified_base (6)..(8): “n” refers to deoxyinosine
cagcannnctctgagcacctgctg

SEQ ID NO: 4 - DNA, 26 bp, Artificial Sequence
  the sequence is synthesized
  modified_base (6)..(8): “n” refers to deoxyinosine
gtctcnnnggtgtagatgagggagac

SEQ ID NO: 5 - DNA, 30 bp, Artificial Sequence
  the sequence is synthesized
  modified_base (6)..(8): “n” refers to deoxyinosine
gctttnnngaactccctgaaaagctaaagc

SEQ ID NO: 6 - DNA, 30 bp, Artificial Sequence
  the sequence is synthesized
  modified_base (6)..(8): “n” refers to deoxyinosine
ccaccnnngttgggctcaaatatacggtgg

SEQ ID NO: 7 - DNA, 31 bp, Artificial Sequence
  the sequence is synthesized
  modified_base (6)..(8): “n” refers to deoxyinosine
gagatnnnttccttactggtcctcacatctc

SEQ ID NO: 8 - DNA, 28 bp, Artificial Sequence
  the sequence is synthesized
  modified_base (6)..(8): “n” refers to deoxyinosine
tgctgnnntcaccggatcatggccagca

SEQ ID NO: 9 - DNA, 28 bp, Artificial Sequence
  the sequence is synthesized
  modified_base (6)..(8): “n” refers to deoxyinosine
tctcannngcttgggtgaaagagtgaga

SEQ ID NO: 10 - DNA, 24 bp, Artificial Sequence
  the sequence is synthesized
  modified_base (6)..(8): “n” refers to deoxyinosine
ggtccnnnggtgggttatgggacc

SEQ ID NO: 11 - DNA, 27 bp, Artificial Sequence
  the sequence is synthesized
  modified_base (6)..(8): “n” refers to deoxyinosine
cgttannncggacagcgatctctaacg

SEQ ID NO: 12 - DNA, 28 bp, Artificial Sequence
  the sequence is synthesized
  modified_base (6)..(8): “n” refers to deoxyinosine
gtaggnnncgtgctcacgttcttcctac

SEQ ID NO: 13 - DNA, 28 bp, Artificial Sequence
  the sequence is synthesized
  modified_base (6)..(8): “n” refers to deoxyinosine
caatgnnngaatatctgggcctacattg

SEQ ID NO: 14 - DNA, 26 bp, Artificial Sequence
  the sequence is synthesized
  modified_base (6)..(8): “n” refers to deoxyinosine
agaaannncactccatcgagatttct

SEQ ID NO: 15 - DNA, 26 bp, Artificial Sequence
  the sequence is synthesized
  modified_base (6)..(8): “n” refers to deoxyinosine
aactgnnngaaaactcggtttcagtt

SEQ ID NO: 16 - DNA, 27 bp, Artificial Sequence
  the sequence is synthesized
  modified_base (6)..(8): “n” refers to deoxyinosine
gtgttnnncaggtaaatcagctaacac

SEQ ID NO: 17 - DNA, 24 bp, Artificial Sequence
  the sequence is synthesized
  modified_base (6)..(8): “n” refers to deoxyinosine
cgtggnnnggttttccctgccacg

SEQ ID NO: 18 - DNA, 26 bp, Artificial Sequence
  the sequence is synthesized
  modified_base (6)..(8): “n” refers to deoxyinosine
ctcctnnnccggagtgtatgcaggag

SEQ ID NO: 19 - DNA, 26 bp, Artificial Sequence
  the sequence is synthesized
  modified_base (6)..(8): “n” refers to deoxyinosine
ggctgnnngggctctcttcttcagtc

SEQ ID NO: 20 - DNA, 25 bp, Artificial Sequence
  the sequence is synthesized
  modified_base (6)..(8): “n” refers to deoxyinosine
gagggnnncagacctctcaaccctc

SEQ ID NO: 21 - DNA, 28 bp, Artificial Sequence
  the sequence is synthesized
  modified_base (6)..(8): “n” refers to deoxyinosine
caggtnnncattcatgcgtcttcacctg

SEQ ID NO: 22 - DNA, 26 bp, Artificial Sequence
  the sequence is synthesized
  modified_base (6)..(8): “n” refers to deoxyinosine
tgcagnnncgttgggcatgagctgta

SEQ ID NO: 23 - DNA, 32 bp, Artificial Sequence
  the sequence is synthesized
  modified_base (6)..(8): “n” refers to deoxyinosine
catctnnnggtctcataagacttcctgagatg

SEQ ID NO: 24 - DNA, 26 bp, Artificial Sequence
  the sequence is synthesized
  modified_base (6)..(8): “n” refers to deoxyinosine
gagttnnncaggctggtctcaaacac

SEQ ID NO: 25 - DNA, 25 bp, Artificial Sequence
  the sequence is synthesized
  modified_base (6)..(8): “n” refers to deoxyinosine
tgggannngctccttcatcgtccca

SEQ ID NO: 26 - DNA, 28 bp, Artificial Sequence
  the sequence is synthesized
  modified_base (6)..(8): “n” refers to deoxyinosine
ggactnnngtccggtattcgcagaagtc

SEQ ID NO: 27 - DNA, 26 bp, Artificial Sequence
  the sequence is synthesized
  modified_base (6)..(8): “n” refers to deoxyinosine
gatccnnngaaggtgtctgcgggatc

SEQ ID NO: 28 - DNA, 26 bp, Artificial Sequence
  the sequence is synthesized
  modified_base (6)..(8): “n” refers to deoxyinosine
tcgatnnngcgtgatgatgaaatcga

SEQ ID NO: 29 - DNA, 31 bp, Artificial Sequence
  the sequence is synthesized
  modified_base (6)..(8): “n” refers to deoxyinosine
ctttcnnngtcatgctgaaagtaggagaaag

SEQ ID NO: 30 - DNA, 32 bp, Artificial Sequence
  the sequence is synthesized
  modified_base (6)..(8): “n” refers to deoxyinosine
tgaaannntagtccacagtgttttcagtttca

SEQ ID NO: 31 - DNA, 24 bp, Artificial Sequence
  the sequence is synthesized
  modified_base (6)..(8): “n” refers to deoxyinosine
cgatgnnncctctgacgtccatcg

SEQ ID NO: 32 - DNA, 26 bp, Artificial Sequence
  the sequence is synthesized
  modified_base (6)..(8): “n” refers to deoxyinosine
gatgcnnntgcagcagtctccgcatc

SEQ ID NO: 33 - DNA, 26 bp, Artificial Sequence
  the sequence is synthesized
  modified_base (6)..(8): “n” refers to deoxyinosine
dcagcnnntgtggtagttggagctgh

SEQ ID NO: 34 - DNA, 30 bp, Artificial Sequence
  the sequence is synthesized
  modified_base (6)..(8): “n” refers to deoxyinosine
gactcnnnccataggtacatcttcagagtc

SEQ ID NO: 35 - DNA, 27 bp, Artificial Sequence
  the sequence is synthesized
  modified_base (6)..(8): “n” refers to deoxyinosine
tgtccnnngtaaagctggaaagggata

SEQ ID NO: 36 - DNA, 29 bp, Artificial Sequence
  the sequence is synthesized
  modified_base (6)..(8): “n” refers to deoxyinosine
ccagtnnncaagatctgaagctctactgg

SEQ ID NO: 37 - DNA, 32 bp, Artificial Sequence
  the sequence is synthesized
  modified_base (6)..(8): “n” refers to deoxyinosine
gcgatnnnttattagagggtggggcggatcgc

SEQ ID NO: 38 - DNA, 29 bp, Artificial Sequence
  the sequence is synthesized
  modified_base (6)..(8): “n” refers to deoxyinosine
ttacgnnngaccccgaaccgcgaccgtaa

SEQ ID NO: 39 - DNA, 28 bp, Artificial Sequence
  the sequence is synthesized
  modified_base (6)..(8): “n” refers to deoxyinosine
cgaatnnntcgagaacgcgagcgattcg

SEQ ID NO: 40 - DNA, 28 bp, Artificial Sequence
  the sequence is synthesized
  modified_base (6)..(8): “n” refers to deoxyinosine
tcgttnnngaccaatccaaccgaaacga

SEQ ID NO: 41 - DNA, 28 bp, Artificial Sequence
  the sequence is synthesized
  modified_base (6)..(8): “n” refers to deoxyinosine
gaccgnnngggtatgcgtcgattcggtc

SEQ ID NO: 42 - DNA, 28 bp, Artificial Sequence
  the sequence is synthesized
  modified_base (6)..(8): “n” refers to deoxyinosine
cgtttnnngcgaaaacgaaaccgaaacg

SEQ ID NO: 43 - DNA, 31 bp, Artificial Sequence
  the sequence is synthesized
  modified_base (6)..(8): “n” refers to deoxyinosine
gacgcnnntggtagtttttatgaaaggcgtc

SEQ ID NO: 44 - DNA, 27 bp, Artificial Sequence
  the sequence is synthesized
  modified_base (6)..(8): “n” refers to deoxyinosine
cgtggnnncctctaaccgcccaccacg

SEQ ID NO: 45 - DNA, 32 bp, Artificial Sequence
  the sequence is synthesized
  modified_base (6)..(8): “n” refers to deoxyinosine
gacgtnnnggatagtcggatcgagttaacgtc

SEQ ID NO: 46 - DNA, 24 bp, Artificial Sequence
  the sequence is synthesized
  modified_base (6)..(8): “n” refers to deoxyinosine
tcggcnnnccctcccaaacgccga

SEQ ID NO: 47 - DNA, 26 bp, Artificial Sequence
  the sequence is synthesized
  modified_base (6)..(8): “n” refers to deoxyinosine
gacccnnntattgcggagtgcgggtc

SEQ ID NO: 48 - DNA, 26 bp, Artificial Sequence
  the sequence is synthesized
  modified_base (6)..(8): “n” refers to deoxyinosine
tcgtcnnntcgacgaactcccgacga

SEQ ID NO: 49 - DNA, 30 bp, Artificial Sequence
  the sequence is synthesized
  modified_base (6)..(8): “n” refers to deoxyinosine
acgcgnnnttaggttagagggttatcgcgt

SEQ ID NO: 50 - DNA, 31 bp, Artificial Sequence
  the sequence is synthesized
  modified_base (6)..(8): “n” refers to deoxyinosine
gtcggnnntaactaaaaattcacctaccgac

SEQ ID NO: 51 - DNA, 32 bp, Artificial Sequence
  the sequence is synthesized
  modified_base (6)..(8): “n” refers to deoxyinosine
gcgacnnnacgtagacgttttattagggtcgc

SEQ ID NO: 52 - DNA, 28 bp, Artificial Sequence
  the sequence is synthesized
  modified_base (6)..(8): “n” refers to deoxyinosine
cgcggnnncctcatcgtaactacccgcg

SEQ ID NO: 53 - DNA, 28 bp, Artificial Sequence
  the sequence is synthesized
  modified_base (6)..(8): “n” refers to deoxyinosine
gatacnnngtgttaacgcgttgcgtatc

SEQ ID NO: 54 - DNA, 29 bp, Artificial Sequence
  the sequence is synthesized
  modified_base (6)..(8): “n” refers to deoxyinosine
tcgttnnnaaccccgcgaactaaaaacga

SEQ ID NO: 55 - DNA, 28 bp, Artificial Sequence
  the sequence is synthesized
  modified_base (6)..(8): “n” refers to deoxyinosine
gaaacnnngtgggcgggtcgttagtttc

SEQ ID NO: 56 - DNA, 34 bp, Artificial Sequence
  the sequence is synthesized
  modified_base (6)..(8): “n” refers to deoxyinosine
cgtcgnnnctcacaaatactttacaattccgacg

SEQ ID NO: 57 - DNA, 28 bp, Artificial Sequence
  the sequence is synthesized
  modified_base (6)..(8): “n” refers to deoxyinosine
gcgaannntcgcggggttcgtttttcgc

SEQ ID NO: 58 - DNA, 30 bp, Artificial Sequence
  the sequence is synthesized
  modified_base (6)..(8): “n” refers to deoxyinosine
cgcgtnnngacgttttcattcatacacgcg

SEQ ID NO: 59 - DNA, 27 bp, Artificial Sequence
  the sequence is synthesized
  modified_base (6)..(8): “n” refers to deoxyinosine
aaccgnnngcgttcgtattttgcggtt

SEQ ID NO: 60 - DNA, 30 bp, Artificial Sequence
  the sequence is synthesized
  modified_base (6)..(8): “n” refers to deoxyinosine
tcggtnnncgtacaataaccgaacgaccga

SEQ ID NO: 61 - DNA, 30 bp, Artificial Sequence
  the sequence is synthesized
  modified_base (6)..(8): “n” refers to deoxyinosine
gcgtannntggaggatttttttgcgtacgc

SEQ ID NO: 62 - DNA, 25 bp, Artificial Sequence
  the sequence is synthesized
  modified_base (6)..(8): “n” refers to deoxyinosine
ttcgcnnngaaccgaacgccgcgaa

SEQ ID NO: 63 - DNA, 32 bp, Artificial Sequence
  the sequence is synthesized
  modified_base (6)..(8): “n” refers to deoxyinosine
taaaannnttggggcgcgggtttgggttttta

SEQ ID NO: 64 - DNA, 31 bp, Artificial Sequence
  the sequence is synthesized
  modified_base (6)..(8): “n” refers to deoxyinosine
tagtgnnncgtaaacgacgccgaccccacta

SEQ ID NO: 65 - DNA, 33 bp, Artificial Sequence
  the sequence is synthesized
  modified_base (6)..(8): “n” refers to deoxyinosine
gacgannnttttgggattgtatttgttttcgtc

SEQ ID NO: 66 - DNA, 32 bp, Artificial Sequence
  the sequence is synthesized
  modified_base (6)..(8): “n” refers to deoxyinosine
cggggnnnaacaaaatacaaaccgtatccccg

SEQ ID NO: 67 - DNA, 34 bp, Artificial Sequence
  the sequence is synthesized
  modified_base (6)..(8): “n” refers to deoxyinosine
gcgcgnnntgtagttttcggagttagtgtcgcgc

SEQ ID NO: 68 - DNA, 33 bp, Artificial Sequence
  the sequence is synthesized
  modified_base (6)..(8): “n” refers to deoxyinosine
cgttcnnncctacgatcgaaaacgacgcgaacg

SEQ ID NO: 69 - DNA, 28 bp, Artificial Sequence
  the sequence is synthesized
  modified_base (6)..(8): “n” refers to deoxyinosine
gaaacnnnggcggttttcggtaggtttc

SEQ ID NO: 70 - DNA, 31 bp, Artificial Sequence
  the sequence is synthesized
  modified_base (6)..(8): “n” refers to deoxyinosine
cgtcgnnngaaacgaaactaacgaaacgacg

SEQ ID NO: 71 - DNA, 36 bp, Artificial Sequence
  the sequence is synthesized
  modified_base (6)..(8): “n” refers to deoxyinosine
cgttannncggtagttttttggttttcgtggtaacg

SEQ ID NO: 72 - DNA, 33 bp, Artificial Sequence
  the sequence is synthesized
  modified_base (6)..(8): “n” refers to deoxyinosine
cgattnnntcaacgaactcacgccgcgcaatcg

(19) United States
(12) Patent Application Publication
(10) Pub. No.: US 2019/0106735 A1
(43) Pub. Date: Apr. 11, 2019

(54) METHOD FOR ANALYZING CANCER GENE USING MULTIPLE AMPLIFICATION NESTED SIGNAL AMPLIFICATION AND KIT

(71) Applicant: DIOGENE CO., LTD., Seongnam-si (KR)

(72) Inventors: Seong-Sik LIM, Namyangju-si (KR); Yeon-Soo KIM, Namyangju-si (KR); Jun KANG, Suwon-si (KR); Jung Suk LIM, Seoul (KR); Se Eun PARK, Seoul (KR)

(21) Appl. No.: 15/981,028

(22) Filed: May 16, 2018

(30) Foreign Application Priority Data
Jul. 31, 2017 (KR) 10-2017-0097039

(51) Int. Cl. C12Q 1/6827; C12Q 1/6806; C12Q 1/6886; C12Q 1/686; C12Q 1/6853; C12Q 1/6851; C07H 21/04

(52) U.S. Cl. CPC C12Q 1/6827; C12Q 1/6806; C12Q 1/6886; C12Q 1/686; C12Q 1/6853; C12Q 2600/16; C07H 21/04; C12Q 2600/154; C12Q 2600/158; C12Q 2600/156; C12Q 2600/112; C12Q 1/6851

(57) ABSTRACT

According to the present disclosure, a cancer gene analysis method comprises performing multiplex polymerase chain reaction (MPCR) on a nucleic acid sample extracted from a biological sample using a cancer gene-specific primer set including four or more species of sequences selected from the group consisting of sequence numbers 1 to 72, performing multiplex real-time polymerase chain reaction (MRTPCR) on a product of the MPCR using the primer set and a marker, and identifying the type of the cancer gene using a product of the MRTPCR. The primer set includes an oligonucleotide of a dumbbell structure represented in the following formula: 5′-Ap-Bq-Cr-3′.

### CROSS-REFERENCE TO RELATED APPLICATIONS

This patent application claims priority under 35 U.S.C. § 119 to Korean Patent Application No. 10-2017-0097039, filed on Jul. 31, 2017, in the Korean Intellectual Property Office, the disclosure of which is incorporated by reference herein in its entirety.

### STATEMENT REGARDING SPONSORED RESEARCH OR DEVELOPMENT

This invention was made with Korean government support under Gyeonggido Technology Development Project (Project No.: D161643; Project Title: DEVELOPMENT OF GENETIC VARIATION TESTING KIT FOR 10 MOST COMMON CANCER TYPES USING MULTIPLE AMPLIFICATION NESTED SIGNAL AMPLIFICATION; Research Period: Sep. 1, 2016 through Aug. 31, 2017;) awarded by Gyeonggido Business & Science Accelerator (“GBSA”). The Korean government has certain rights in the invention.

### TECHNICAL FIELD

Embodiments of the present disclosure are directed to genetic analysis techniques, and more specifically, to methods and kits for analyzing cancer genes or determining occurrence or development of mutation or methylation in genes.

### DISCUSSION OF RELATED ART

Cancer is a group of diseases involving abnormal cell growth with the potential to invade or spread to other parts of the body. Cancer can spread from its original site by local spread, lymphatic spread to regional lymph nodes or by hematogenous spread via the blood to distant sites, known as metastasis. When cancer spreads by a hematogenous route, it usually spreads all over the body. However, cancer ‘seeds’ grow in certain selected site only (‘soil’) as hypothesized in the soil and seed hypothesis of cancer metastasis. The cancer burden can also be reduced through early detection of cancer and management of patients who develop cancer. Many cancers have a high chance of cure if diagnosed early and treated adequately. Various research and development efforts are underway for early diagnosis of cancer.

It has been known that abnormally is found from particular genes right before cells turn into cancer cells and that DNA methylation or demethylation influences the occurrence of cancer. There are ongoing vigorous research made worldwide to enable early detection of cancer based on such knowledge, but no satisfactory achievements have been brought up yet.

### SUMMARY

According to the present disclosure, a cancer gene analysis method comprises performing multiplex polymerase chain reaction (MPCR) on a nucleic acid sample extracted from a biological sample using a cancer gene-specific primer set including four or more species of sequences selected from the group consisting of sequence numbers 1 to 72, performing multiplex real-time polymerase chain reaction (MRTPCR) on a product of the MPCR using the primer set and a marker, and identifying the type of the cancer gene using a product of the MRTPCR. The primer set includes an oligonucleotide of a dumbbell structure represented in the following formula: 5′-Ap-Bq-Cr-3′, where Ap is a specificity portion having a hybridization sequence continuous and complementary to the 3′, Bq is a separation portion including three or more universal bases, Cr is a variation specificity portion substantially joined to a target sequence, and p, q, and r each are the number of nucleotides.

According to an embodiment of the present disclosure, p and q each are an integer from 3 to 5, and r is an integer from 18 to 30.

According to an embodiment of the present disclosure, the MPCR includes preliminary modification at 95° C. for 10 minutes, repeating 30 times a series of steps including modification at 95° C. for 30 seconds, annealing at 62° C. for one minute, and expansion at 72° C. for one minute, and amplification at 72° C. for five minutes.

According to an embodiment of the present disclosure, the MRTPCR includes preliminary modification at 95° C. for 10 minutes and repeating 30 times a series of steps including modification at 95° C. for 20 seconds, annealing at 62° C. for 20 seconds, and expansion at 72° C. for 35 seconds.

According to an embodiment of the present disclosure, the marker includes one or more selected from the group consisting of FAM (5-carboxy fluorescein), ROX (carboxy-Xrhodamine), HEX (hexachlorofluorescein), Cal Fluor Red 610 (C46H57F6N5O4P2), Cy5 (cyanine-based fluorescent substance), Cy3, TAMRA (5-Carboxytetramethylrhodamine), Alexa 532, Rhodamine, tetra methyl rhodamine, Oregon green, R-PE, Alexa 546, Bodipy-TMRX, PBXL-1, Texas Red. Cryptofluor Orange, cyanine-based dye, and biotin.

According to an embodiment of the present disclosure, identifying the type of the cancer gene includes heating the product of the MPCR at an interval of 4° C. to 5° C. per minute to 65° C. to 95° C. and analyzing a melting temperature curve according to a fluorescent signal.

According to an embodiment of the present disclosure, a method for amplifying a cancer gene comprises performing multiplex polymerase chain reaction (MPCR) on a nucleic acid sample extracted from a biological sample using a cancer gene-specific primer set including four or more species of sequences selected from the group consisting of sequence numbers 1 to 72, screening the cancer gene by identifying a product of the MPCR, wherein the primer set includes an oligonucleotide of a dumbbell structure represented in Formula 1, wherein [Formula 1] 5′-Ap-Bq-Cr-3′, wherein Ap is a specificity portion having a hybridization sequence continuous and complementary to the 3′, Bq is a separation portion including three or more universal bases, Cr is a variation specificity portion substantially joined to a target sequence, and p, q, and r each are the number of nucleotides.

According to an embodiment of the present disclosure, a kit for analyzing a cancer gene comprises a primer set for analyzing the cancer gene, a dNTP mixture, a DNA polymerase, and a marker, wherein the primer set includes four or more species of sequences selected from the group consisting of sequence numbers 1 to 72, wherein the primer set includes an oligonucleotide of a dumbbell structure represented in Formula 1, wherein [Formula 1] 5′-Ap-Bq-Cr-3′, wherein Ap is a specificity portion having a hybridization sequence continuous and complementary to the 3′, Bq is a separation portion including three or more universal bases, Cr is a variation specificity portion substantially joined to a target sequence, and p, q, and r each are the number of nucleotides.

According to an embodiment of the present disclosure, the kit is used to simultaneously analyze gene mutation and methylation related to one or more species of cancer selected from the group consisting of liver cancer, lung cancer, bladder cancer, gastric cancer, breast cancer, uterine cancer, colon cancer, colorectal cancer, blood cancer, ovary cancer, and prostate cancer.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a view illustrating an example of performing an MPCR followed by a MRPPCR in a method for analyzing a cancer gene according to an embodiment of the present disclosure;

FIG. 2 is a table illustrating results of single-time PCR analysis to check on amplification capacity for gene mutation and methylation using each positive control substance and each negative control substance according to an embodiment of the present disclosure;

FIG. 3 is a table illustrating melting temperature curves obtained by detecting signals generated at cycles to analyze sensitivity of products PCRed by MPCRed products, according to embodiment 3 of the present disclosure;

FIG. 4 is a table illustrating results of analysis of melting temperature curves for 18 species of gene mutations of products PCRed using products obtained by MPCRing DLP primers according to embodiment 5 of the present disclosure; and

FIG. 5 is a table illustrating results of analysis of melting temperature curves for 18 species of methylated genes of products PCRed using products obtained by MPCRing DLP primers according to embodiment 5 of the present disclosure.

### DETAILED DESCRIPTION OF EXEMPLARY EMBODIMENTS

Hereinafter, exemplary embodiments of the present disclosure will be described in detail with reference to the accompanying drawings. The same reference denotations may be used to refer to the same or similar elements throughout the specification and the drawings. The present disclosure, however, may be modified in various different ways, and should not be construed as limited to the embodiments set forth herein. As used herein, the singular forms “a,” “an,” and “the” are intended to include the plural forms as well, unless the context clearly indicates otherwise.

As used herein, the term “multiplex PCR amplification” may refer to simultaneously amplyfing multiple genes through a single PCR.

As used herein, the term “multiple amplitude nested signal amplification” (hereinafter, simply referred to as “MANSA”) refers to a method for analyzing the type of a cancer gene, various gene mutations or methylation which is related to the cause of cancer, by a series of processes of simultaneously performing first multiplex PCR amplification (hereinafter, “MPCR”) on multiple target nucleic acids in a multiplexing scheme to obtain a product of the MPCR, performing, real-time, second MPCR on the obtained product of the first MPCR with a marker-containing primer set to detect a target nucleic acid sequence, and checking on the melting temperature curve of the product of the second MPCR.

Hereinafter, embodiments of the present disclosure are described in detail.

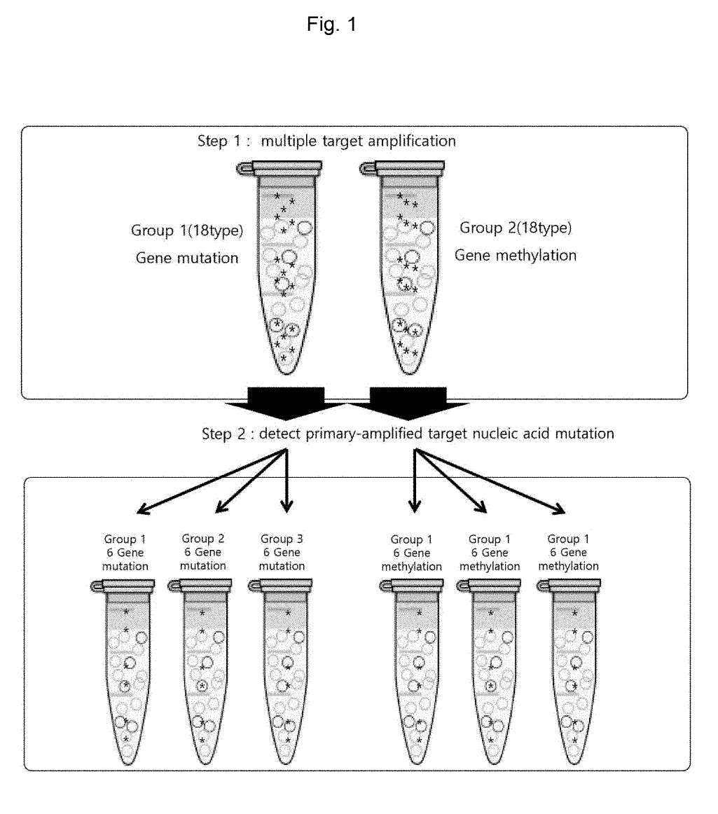

FIG. 1 is a view illustrating an example of performing an MPCR followed by a MRPPCR in a method for analyzing a cancer gene according to an embodiment of the present disclosure.

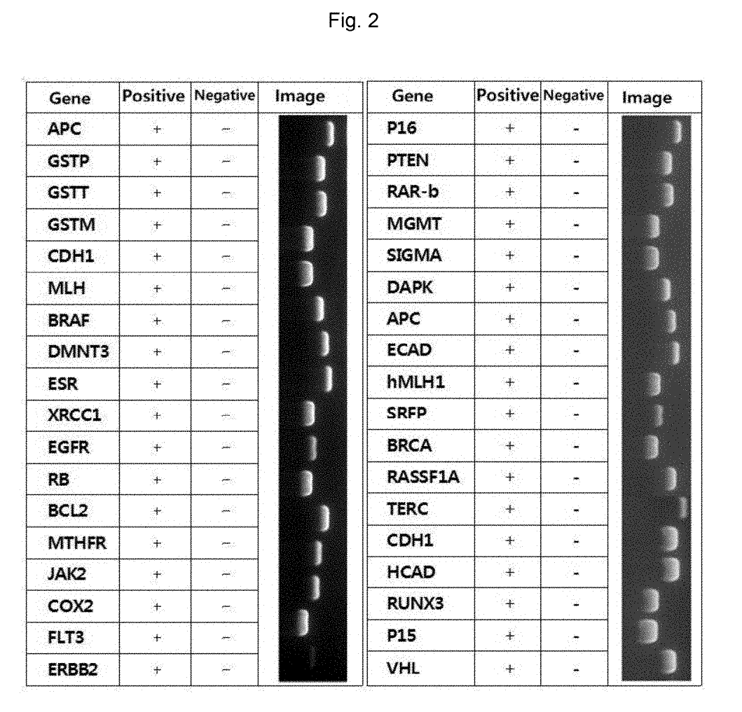

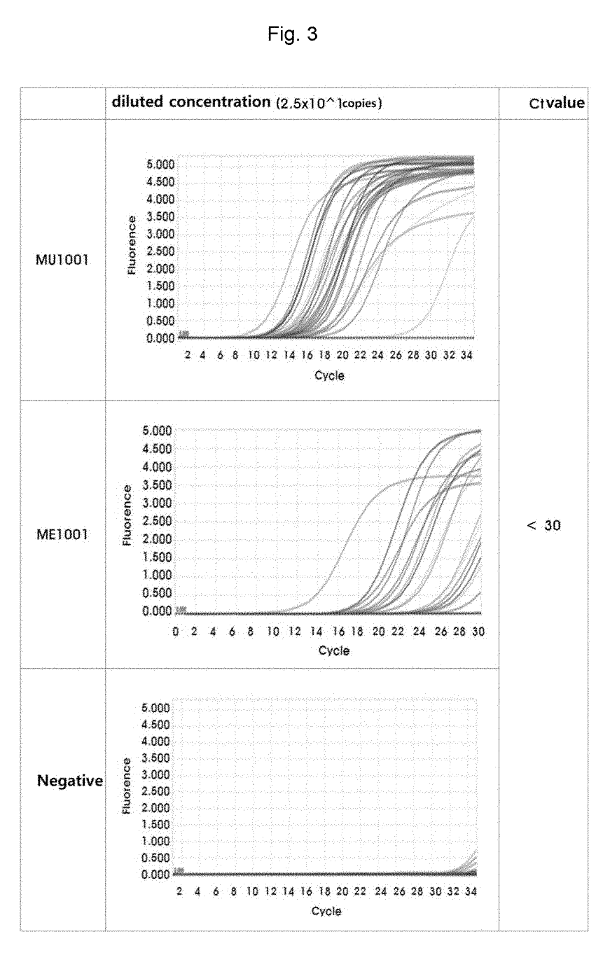

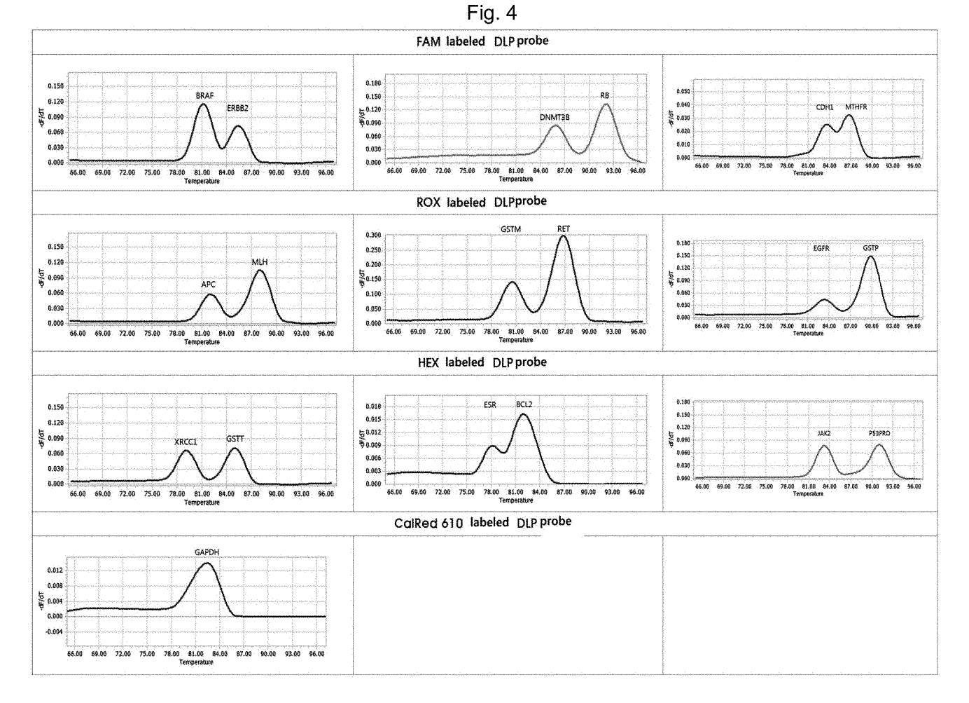

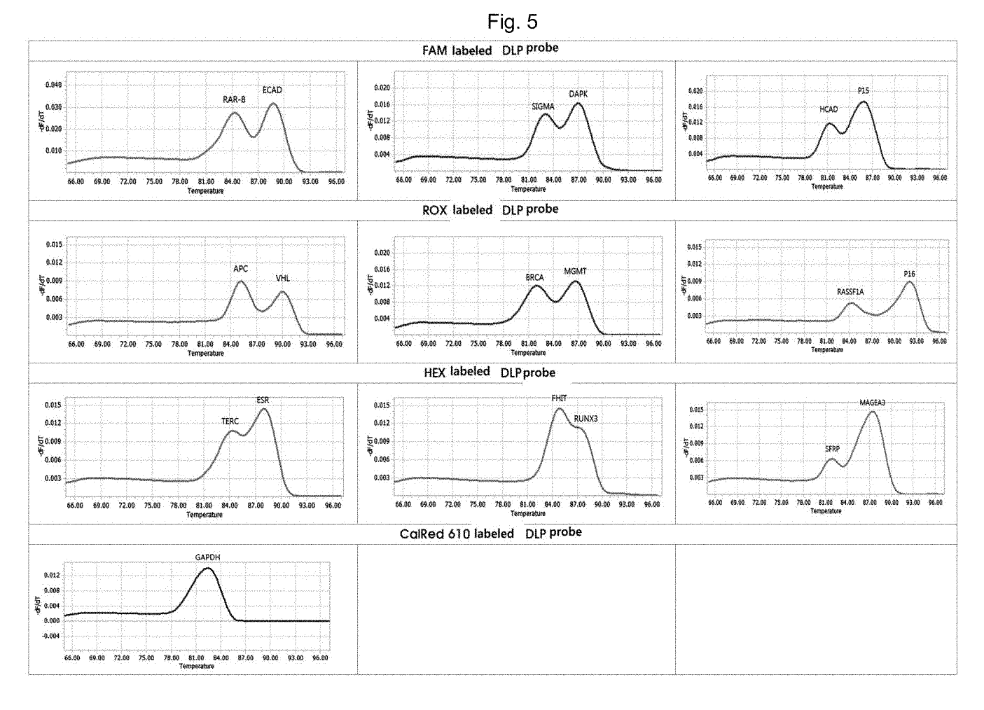

FIG. 2 is a table illustrating results of single-time PCR analysis to check on amplification capacity for gene mutation and methylation using each positive control substance and each negative control substance according to an embodiment of the present disclosure. FIG. 3 is a table illustrating melting temperature curves obtained by detecting signals generated at cycles to analyze sensitivity of products PCRed by MPCRed products, according to embodiment 3 of the present disclosure. FIG. 4 is a table illustrating results of analysis of melting temperature curves for 18 species of gene mutations of products PCRed using products obtained by MPCRing DLP primers according to embodiment 5 of the present disclosure. FIG. 5 is a table illustrating results of analysis of melting temperature curves for 18 species of methylated genes of products PCRed using products obtained by MPCRing DLP primers according to embodiment 5 of the present disclosure.

According to the present disclosure, there is provided a primer set containing dumbbell-structure oligonucleotides each represented in Formula 1 to utilize a method for analyzing a cancer gene and a cancer gene analysis kit for the same which is described below.

5′-Ap-Bq-Cr-3′ [Formula]

Each primer of the primer set includes three separation portions Ap, Bq, and Cr as shown above. The basic structure of the primer including the oligonucleotide with the above structure may be named a dumbbell structure specificity structure. Hereinafter, primers with such a structure are collectively referred to as dumbbell likely primers (DLPs).

The primer is a brand-new primer developed by the inventors. The primer may exhibit significantly enhanced hybridization specificity because the hybridization is type-specifically determined by the 5′-low Tm specificity portion and the 3′-high Tm specificity portion separated by the separation portion, and this primer may have a structure including a continuously complementary hybridization sequence, separation portions, and specificity portions.

Gene amplification using the oligonucleotide-containing primer set may suppress unintentional PCR amplification of genes from occurring at room temperature, thereby enabling hot-start PCR. This way may also allow amplification based on the PCRed product to be dominant over amplification using the first mold with the result of suppressing non-specific amplification and overall enhancement in the PCT specificity.

Specifically, in Formula 1 above, Ap is a specificity portion having the hybridization sequence continuously complementary to the 3′, Bq is a separation portion including three or more universal bases, Cr is a variation specificity portion which is substantially joined to a target sequence, and p, q, and r refer to the number of the nucleotide bases. Ap, which is positioned adjacent to the variation specificity portion Cr, may be referred to as a variation adjacent specificity portion. Preferably, p may be an integer from 3 to 5, q may be an integer from 3 to 5, and r may be an integer from 18 to 30.

The separation portion includes continuous universal bases. The variation adjacent specificity portion Ap may be shorter than the variation specificity portion Cr. The variation adjacent specificity portion may have a length of, preferably, three to 15 nucleotides, more preferably five and six nucleotides. The variation specificity portion may have a length of, preferably, three to 20 nucleotides, more preferably five to 19 nucleotides, most preferably six to 18 nucleotides. The separation portion preferably has a length of three to five nucleotides.

The universal bases in the separation portion of the primer may be selected from the group consisting of deoxyinosine, inosine, 7-deaza-2′-deoxyinosine, 2-aza-2′-deoxyinosine, 2′-OMe inosine, 2′-F inosine, and a combination thereof. More preferably, the universal base may be deoxyinosine, inosine, or 5-nitroindole. Most preferably, the universal base may include deoxyinosine.

The variation adjacent specificity portion of the primer has a Tm of 10° C. to 35° C., more preferably of 5° C. to 12° C. Preferably, the variation specificity portion has a Tm of 50° C. to 65° C. The separation portion preferably has a Tm of 3° C. to 10° C.

The variation specificity portion of the primer includes nucleotides complementary or corresponding to nucleotide variations. Where the primer is hybridized with the sense strand of target nucleotides, the variation specificity portion includes nucleotides complementary to nucleotide variations, and where the primer is hybridized with the anti-sense strand, the variation specificity portion includes nucleotides corresponding to nucleotide variations.

The nucleotide complementary or corresponding to the nucleotide variation is positioned at the 3′ terminus of the variation specificity portion or one or two bases away from the 3′ terminus of the variation specificity portion, more preferably one or two bases away from the 3′ terminus of the variation specificity portion, most preferably at or near the middle of the variation specificity portion. As an example, where the variation specificity portion is one or two bases away from the 3′ terminus, the nucleotide complementary or corresponding to the nucleotide variation is positioned at the 5′ terminus of the variation specificity portion or one or two bases away from the 5′ terminus of the variation specificity portion.

As the nucleotide corresponding to the nucleotide variation is positioned one or two bases away from the 3′ terminus of the variation specificity portion and the 5′ terminus of the complementary nucleotide variation specificity portion is positioned one or two bases away, the following advantageous effects are presented.

For example, when single nucleotide polymorphism (SNP) is detected using normal primers, the varied portion is positioned at the 3′ terminus in which case the Tm value does not make a significant difference between when the base of the 3′ terminus is annealed to the target sequence and when the base of the 3′ terminus is not (i.e., mismatches). Thus, despite a mismatch, annealing and resultantly amplification reaction may arise, ending up with the result of being false positive. In contrast, when the varied portion is positioned at the middle, the Tm value shows a difference to some degree between when the varied portion is annealed to the target sequence and when it is not (i.e., a mismatch occurs). However, most of thermostable polymerases catalyze even the mismatched portion for polymerization, giving the result of being false positive.

The cancer gene analysis method of the present disclosure may fully address the problems of the related art. For example, if a mismatch occurs in a case where the nucleotide complementary or corresponding to the nucleotide variation is positioned at the middle of the variation specificity portion, the mismatch may be taken as having occurred at the center of the structure in light of the variation specificity portion alone. Thus, the Tm value of the variation specificity portion is sharply reduced as compared with when matched. From a point of view of the overall primer structure, mismatches simultaneously occur at the 5′ terminus and the 3′ terminus, and thus, thermostable polymerases do not conduct catalysis. Thus, upon mismatching, no result of being false positive occurs.

Accordingly, the above-structured primer, when annealed to the nucleotide variation-containing target sequence, is determined for its specificity not by its overall length as are conventional primers but by the variation adjacent specificity portion and variation specificity portion, which are separated from each other. Thus, the primer is annealed to the target sequence in a different manner from conventional primers, and this leads to a significant enhancement in the annealing specificity.

To that end, the primer set contains 36 species of oligonucleotides designed specific to cancer genes. The oligonucleotides may include primers having four or more species of sequences selected from the group consisting of sequence numbers 1 to 72 as shown in Tables 1 and 2 below. Sequences from sequence number 1 to sequence number 72 are disclosed herein. In Tables 1 and 2, I refers to deoxyinosine, I refers to deoxyinosine, and the bold letters (e.g., T) denote the positions where fluorescent colors are joined for signal amplification.)

TABLE 1

| Sequence | | | Sequence | | |
|---|---|---|---|---|---|
| Number | Gene | forward primer | Number | Gene | reverse primer |
| 1 | APC | GAGGTIIICCACACAGAACTACCTC | 2 | APC | GGTTIIITTATGAGAAAAGCAAACC |
| 3 | GSTP | CAGCAIIICTCTGAGCACCTGCTG | 4 | GSTP | GTCTCIIIGGTGTAGATGAGGGAGAC |
| 5 | GSTT | GCTTTIIIGAACTCCCTGAAAAGCTAAAGC | 6 | GSTT | CCACCIIIGTTGGGCTCAAATATACGGTGG |
| 7 | GSTM | GAGATIIITTCCTTACTGGTCCTCACATCTC | 8 | GSTM | TGCTGIIITCACCGGATCATGGCCAGCA |
| 9 | CDH1 | TCTCAIIIGCTTGGGTGAAAGAGTGAGA | 10 | CDH1 | GGTCCIIIGGTGGGTTATGGGACC |
| 11 | MLH | CGTTAIIICGGACAGCGATCTCTAACG | 12 | MLH | GTAGGIIICGTGCTCACGTTCTTCCTAC |
| 13 | BRAF | CAATGIIIGAATATCTGGGCCTACATTG | 14 | BRAF | AGAAAIIICACTCCATCGAGATTTCT |
| 15 | DMNT3 | AACTGIIIGAAAACTCGGTTTCAGTT | 16 | DMNT3 | GTGTTIIICAGGTAAATCAGCTAACAC |
| 17 | ESR | CGTGGIIIGGTTTTCCCTGCCACG | 18 | ESR | CTCCTIIICCGGAGTGTATGCAGGAG |
| 19 | XRCC1 | GGCTGIIIGGGCTCTCTTCTTCAGTC | 20 | XRCC1 | GAGGGIIICAGACCTCTCAACCCTC |
| 21 | EGFR | CAGGTIIICATTCATGCGTCTTCACCTG | 22 | EGFR | TGCAGIIICGTTGGGCATGAGCTGTA |
| 23 | RB | CATCTIIIGGTCTCATAAGACTTCCTGAGATG | 24 | RB | GAGTTIIICAGGCTGGTCTCAAACAC |
| 25 | BCL2 | TGGGAIIIGCTCCTTCATCGTCCCA | 26 | BCL2 | GGACTIIIGTCCGGTATTGGCAGAAGTC |
| 27 | MTHFR | GATCCIIIGAAGGTGTCTGCGGGATC | 28 | MTHFR | TCGATIIIGCGTGATGATGAAATCGA |
| 29 | JAK2 | CTTCIIIGTCATGCTGAAAGTAGGAGAAAG | 30 | JAK2 | TGAAGIII TAGTCCACAGTGTTTTCAGTTTCA |
| 31 | ERBB2 | CGATGIIICCTCTGACGTCCATCG | 32 | ERBB2 | GATGCIIITGCAGCAGTCTCCGCATC |
| 33 | KRAS1 | DCACGCIIITGTGGTAGTTGGAGCTGH | 34 | KRAS1 | GACTCIIICCATAGGTACATCTTCAGAGTC |
| 35 | PTEN | TGTCCIIIGTAAAGCTGGAAAGGGATA | 36 | PTEN | CCAGTIIICAAGATCTGAAGCTCTACTGG |

TABLE 2

| Related | Sequence | | Sequence | |
|---|---|---|---|---|
| Gene | Number | forward primer | Number | reverse primer |
| P16 | 37 | GCGATIIITTATTAGAGGGTGGGGCGGATCGC | 38 | TTACGIIIGACCCCGAACCGCGACCGTAA |
| RAR-b | 39 | CGAATIIITCGAGAACGCGAGCGATTCG | 40 | TCGTTIIIGACCAATCCAACCGAAACGA |
| MGMT | 41 | GACCGIIIGGGTATGCGTCGATTCGGTC | 42 | CGTTTIIIGCGAAAACGAAACCGAAACG |
| SIGMA | 43 | GACGCIIITGGTAGTTTTTATGAAAGGCGTC | 44 | CGTGGIIICCTCTAACCGCCCACCACG |
| DAPK | 45 | GACGTIIIGGATAGTCGGATCGAGTTAACGTC | 46 | TCGGCIIICCCTCCCAAACGCCGA |
| APC | 47 | GACCCIIITATTGCGGAGTGCGGGTC | 48 | TCGTCIIITCGACGAACTCCCGACGA |
| Ecad | 49 | ACGCGIIITTAGGTTAGAGGGTTATCGCGT | 50 | GTCGGIIITAACTAAAAATTCACCTACCGAC |
| hMLH1 | 51 | GCGACIIIACGTAGACGTTTTATTAGGGTCGC | 52 | CGCGGIIICCTCATCGTAACTACCCGCG |
| RASSF1A | 53 | GATACIIIGTGTTAACGGGTTGCGTATC | 54 | TCGTTIIIAACCCCGCGAACTAAAAACGA |
| CDH1 | 55 | GAAACIIIGTGGGCGGGTCGTTAGTTTC | 56 | CGTCGIIICTCACAAATACTTTACAATTCCGAG |
| HCAD | 57 | GCGAAIIITCGCGGCCTTCGTTTTTCGC | 58 | CGCGTIIIGACGTTTTCATTCATACACGCG |
| P15 | 59 | AACCGIIIGCGTTCGTATTTTGCGGTT | 60 | TCGGTIIICGTACAATAACCGAACGACCGA |
| VHL | 61 | GCGTAIIITGGAGGATTTTTTTGCGTACGC | 62 | TTCGCIIIGAACCGAACGCCGCGAA |
| FHIT | 63 | TAAAAIIITTGGGGCGCGGGTTTGGGTTTTTA | 64 | TAGTGIIICGTAAACGACGCCGACCCCACTA |
| ESR1 | 65 | GACGAIIITTTTGGGATTGTATTTGTTTTCGTC | 66 | CGGGGIIIAACAAAATACAAACCGTATCCCCG |
| SRFP | 67 | GCGCGIIITGTAGTTTTCGGAGTTAGTGTCGCGC | 68 | CGTTCIIICCTACGATCGAAAACGACGCGAACG |
| RUNX3 | 69 | GAAACIIIGGCGGTTTTCGGTAGGTTTC | 70 | CGTCGIIIGAAACGAAACTAACGAAACGACG |
| BRCA | 71 | CGTTAIIICGGTAGTTTTTTGGTTTTCGTGGTAACG | 72 | CGATTIIITCAACGAACTCACGCCGCGCAATCG |

The forward and reward primers of sequence numbers 1 to 36 as shown in Table 1 above are varied primers designed to be able to detect a mutation, e.g., single nucleotide polymorphism (SNP), in a cancer-related gene from a nucleic acid sample extracted from a biological sample. The forward and reverse primers of sequence numbers 37 to 72 shown in Table 2 above are varied primers designed to be able to detect methylation in a cancer-related gene from a nucleotide sample extracted from a biological sample. Such primers present a dumbbell structure specificity structure, enabling simultaneous amplification of multiple genes by one polymerase chain reaction.

As the primer set containing the forward and reverse primers of sequence numbers 1 to 72 having the above structure includes primers that may be joined specifically to each gene variation known to cause cancer or suppress cancer genes and that function to amplify the gene variations through polymerase chain reaction, the primer set may be hybridized or annealed to the genes mutated or methylated in the nucleotide sequences included in the biological sample using the genes used as molds, forming a dumbbell structure and enabling amplification by MPCR.

According to the present disclosure, multiple target nucleotides may simultaneously be detected in a multiplexing scheme using a primer set containing oligonucleotides of the above structure. The multiple target nucleic acid sequences may be classified and screened to be suited for the purpose of analysis. According to the present disclosure, the use of a tiny amount of probes and samples enables detection of multiple target nucleic acid sequences with better sensitivity as compared with conventional real-time PCR methods. For the purpose, there has been developed the technique of obtaining a product of amplification by first performing multiplex target amplification and then detecting target nucleic acid sequences with the above advantages.

There are no conventional ways developed to be able to simultaneously two or more species of variations from the same bases or same codons or two or more nucleotide variations only with a simple amplification reaction (e.g., PCR). According to the present disclosure, there is a provided a real method for simultaneously detecting such variations only with a simple amplification reaction using an oligonucleotide-containing primer set as described above.

The use of the cancer gene analysis method of the present disclosure enables simultaneous detection of various nucleotide variations or single nucleotide polymorphism (SNP) in nucleotide sequences.

According to the present disclosure, a cancer gene analysis method using a primer set as described above includes the step (a) of performing a multiplex polymerase chain reaction (MPCR) on a nucleic acid sample extracted from a biological sample using a cancer gene-specific primer set containing four or more species of sequences selected from the group consisting of sequence numbers 1 to 72, the step (b) of performing a multiplex real-time polymerase chain reaction (MRTPCR) on a product of the MPCR using the primer set and a marker, and the step (c) of identifying the type of the cancer gene using a product of the MRTPCR.

Step (a) may be the step of amplifying, by an MPCR method, the nucleic acid sample extracted from the biological sample using the primer set containing cancer gene-specific primers including four or more species of sequences selected from the group consisting of sequence numbers 1 to 72.

The oligonucleotides contained in the primer set may be primers that are joined specific to each gene variation (or mutation) known to cause cancer or suppress cancer genes and that amplify the gene variations through PCR, and the oligonucleotides may be hybridized or annealed to the nucleic acid sequences contained in the nucleic acid sample using the nucleic acid sequences as molds, forming a dumbbell structure and enabling amplification by MPCR.

Various biological samples are not limited to specific ones as long as the nucleic acid is obtained from various typical biological samples including, e.g., blood, cells, tissues, saliva, or other secretions. In particular, a nucleic acid sample obtained from a biological sample, such as the blood of a patient who is suffering, or is predicted to suffer, from cancer may be put to use for the MPCR amplification.

One extracted by various typical nucleic acid separation methods may be used as the nucleic acid sample. The nucleic acid sample includes DNA or RNA for simultaneously analyzing multiple target nucleic acid sequences in a multiplexing method. The nucleic acid sample may include double-strained or single-strained DNA, preferably double-strained DNA. When the nucleic acid sample is mRNA, the mRNA may be obtained by producing double-stranded DNA through reverse transcription. The reverse transcription may be performed by a reverse transcriptase with RNase H activity.

The MPCR may be performed using a mixture of the primers, nucleic acid samples, a PCR buffer, deoxynucleotide triphosphates, (dNTPs), and a DNA polymerase.

In this step, the MPCR may be performed by performing preliminary modification (which may be referred to as ‘predenaturation’) at 91° C. to 97° C. for 5 minutes to 20 minutes and repeating 20 times to 50 times a series of steps including modification (which may be referred to as ‘denaturation’) at 91° C. to 97° C. for 10 seconds to 120 seconds, annealing at 50° C. to 65° C. for 30 seconds to 120 seconds, and expansion at 65° C. to 75° C. for 30 seconds to 120 seconds, followed by final expansion and amplification at 65° C. to 75° C. for one minute to 10 minutes. Preferably, the MPCR process may be performed by conducting preliminary modification at 95° C. for 10 minutes and repeating 30 times a series of steps including modification at 95° C. for 30 seconds, annealing at 62° C. for one minute, and expansion at 72° C. for one minute, followed by final expansion and amplification at 72° C. for five minutes.

Also in the instant step, the occurrence or development of cancer may be identified using a real-time PCR analysis method that adopts a real-time gene analysis apparatus. e.g., the LightCycler 96. Specifically, the real-time gene analysis apparatus may perform real-time quantitative and qualitative analysis in the log phase during which the reaction lasts. Thus, use of the apparatus enables determination of whether the nucleic acids contained in the sample are amplified and resultantly an easier check on whether the antioncogene is mutated or methylated. By so doing, the occurrence or development of cancer may easily be validated.

To that end, in the instant step, the primers of sequence numbers 1 to 36 contained in the primer set may be divided into three groups each of which includes six species to detect a variation or mutation, e.g., single nucleotide polymorphism (SNP), in the nucleic acids first amplified. The primers of sequence numbers 37 to 72 contained in the primer set may be divided into three groups each of which includes six species to detect the methylation in the first amplified target nucleic acids. When one or more genes in each group are amplified in this step, the genes related to suppressing or causing cancer may be determined to have been mutated or methylated, enabling early diagnosis of the occurrence or development of cancer.

Specifically, the primers of sequence numbers 1 to 36 may be grouped into 18 primer pairs each having an odd-numbered primer and an even-numbered primer, e.g., a primer with sequence number 1 and another primer with sequence number 2, or a primer with sequence number 3 and another with sequence number 4. The 18 primer pairs may be bundled up into three groups, e.g., a first group of primer pairs with sequence numbers 1 to 12, a second group of primer pairs with sequence numbers 13 to 24, and a third group of primer pairs with sequence numbers 25 to 36. Each group of primer pairs may be put in a tube, subject to PCR amplification. Substantially the same process may also be performed on the primers with sequence numbers 37 to 72.

Step (b) is the step of amplifying the MPCR product obtained in step (a) by a multiplex real-time polymerase chain reaction (MRTPCR) method using the primer set and a marker. This step may convert the MPCR product into single-strand DNA and may be used to analyze occurrence or development through the single-strand DNA.

To that end, in this step, a mixture of the MPCR product, the primer set of sequence numbers 1 to 72, a marker, a PCR buffer, dNTPs, and a DNA polymerase may be prepared to perform the MRTPCR.

In particular, the marker may be joined to a particular portion of the 3′ terminus of each primer of the primer set to enhance the sensitivity and specificity of single-molecule fluorescence spectroscopy.

As the marker, FAM (5-carboxy fluorescein), ROX (carboxy-Xrhodamine). HEX (hexachlorofluorescein), Cal Fluor Red 610(C46H57F6N5O4P2), Cy5 (cyanine-based fluorescent substance), Cy3, TAMRA (5-Carboxytetramethylrhodamine), Alexa 532, rhodamine, tetra methyl rhodamine, Oregon green. R-PE, Alexa 546, Bodipy-TMR-X, PBXL-I, Texas red, cryptofluor orange, cyanine-based dye, biotin, or a mixture thereof may be used.

The MRTPCR may be performed by performing preliminary modification (which is also referred to as ‘predenaturation’) at 91° C. to 97° C. for 5 minutes to 20 minutes and repeating 30 times a series of steps including modification (which is also referred to as ‘denaturation’) at 91° C. to 97° C. for 10 seconds to 60 seconds, annealing at 50° C. to 65° C. for 10 seconds to 60 seconds, and expansion at 65° C. to 75° C. for 10 seconds to 60 seconds. Preferably, the MRTPCR process may be performed by preliminary modification at 95° C. for 10 minutes and repeating 30 times a series of steps including modification at 95° C. for 20 seconds, annealing at 62° C. for 20 seconds, and expansion at 72° C. for 35 seconds.

Step (c) is the step of identifying the type of cancer gene, along with mutation or methylation using the MRTPCR product obtained in step (b). In this step, when a fluorescence signal is detected from the MRTPCR product through the marker, the target nucleic acid sequence is determined to exist in the biological sample, enabling early detection of the occurrence or development of cancer.

The detection of fluorescence signals enables earlier detection of occurrence or development of cancer within a short time by identifying the type of cancer gene in such a manner as to detect, at each cycle, the fluorescence signal from the marker on the MRTPCR product amplified by the MRTPCR using the marker-containing primer set or to analyze the melting temperature curve obtained by heating the MRTPCR product.

The cancer gene analysis method of the present disclosure as set forth above may overcome the limitations and minimize the shortcomings of the conventional false positive primer methods by amplifying the type-specific portions using MPCR and detecting the MPCR product through secondary signal amplification in analyzing the type of cancer gene. The above-described cancer gene analysis through the above-described MANSA process produces very high sensitivity and specificity as compared with existing analysis methods, enabling precise diagnosis of occurrence or development of cancer.

To that end, in this step, the same gene analysis apparatus as that described above may be used to identify the occurrence or development of cancer in such a manner as to analyze detected signals or compare per-cancer gene type melting temperature curves using the same real-time gene analysis apparatus as that described above.

In this case, a minimum detection threshold may be set as a reference for the cancer screening test. For example, when a value in excess of the minimum detection threshold is detected from the sample, it may be determined positive. In this way, various types of cancer genes may easily be determined.

The MRTPCR product may be heated to 65° C. to 95° C. at every 4° C. to every 5° C. per minute, and the values on the melting temperature curve may be analyzed to identify the type of the amplified cancer gene. Thus, early-stage diagnosis of cancer is possible.

The cancer gene analysis method of the present disclosure, as described above, may simultaneously amplify a plurality of genes using the primers or their set (primer set) designed cancer-gene specifically and completely exclude an error that may arise due to being false positive even when a tiny amount of samples is in use. Further, the present method may determine whether cancer genes are mutated and methylated in such a manner as to simultaneously performing MPCR on a plurality of target nucleic acids and followed by second amplifying the MPCR product by the MANSA method to identify fluorescence signals from the marker. Further, the present method may identify the presence of multiple gene variations that may frequently occur in various cancer tissues to determine whether the cancer genes involving a particular cancer have been mutated, thereby enabling quick and precise detection of the occurrence or development of cancer.

The cancer gene analysis method by the MANSA of the present disclosure provides a noticeable approach to overcome the problems with the techniques for applying high throughputs of real-time PCR equipment and detection of multiple target genes, save costs for analysis by curtailing the usage of fluorescent double-marked probes and the amount of samples and time of analysis required, and significantly enhance detection sensitivity.

The cancer gene analysis method of the present disclosure enables simultaneous analysis of gene mutation and methylation related to various types of cancer which include, among others, liver cancer, lung cancer, bladder cancer, gastric cancer, breast cancer, uterine cancer, colon cancer, colorectal cancer, blood cancer, ovarian cancer, and prostate cancer.

According to the present disclosure, there is provided a cancer gene amplification method including the step (1) of performing an MPCR on a nucleic acid sample separated from a biological sample using a cancer gene-specific primer set containing four or more species of sequences selected from the group consisting of sequence numbers 1 to 72 and the step (s) of identifying an MPCR product obtained in step (1).

Step (1) is the step of amplifying the nucleic acid sample extracted from the biological sample using the primer set by an MPCR method.

In step (1), the PCR may be performed by various typical PCR methods.

In a case where the nucleic acid sample contains the gene sequences of mutated or methylated antioncogenes, they are amplified by the primer set, and upon detecting the product of amplification, the plurality of cancer genes may easily be screened, and cancer may be determined to have occurred or be developing. Thus, the use of the type-specific primer set prepared according to the present disclosure enables amplification of various types of cancer genes mutated or methylated, through one PCR process.

According to the present disclosure, there is provided a cancer gene analysis kit that includes a cancer gene analysis primer set containing four or more species of sequences selected from the group consisting of sequence numbers 1 to 72, a dNTP mixture, a DNA polymerase, and a marker.

The cancer gene analysis kit enables checks on whether 36 different species of cancer genes are mutated or methylated, allowing the same to be utilized as early detection of various types of cancer, particularly for simultaneous analysis of gene mutation and methylation related to such cancers as, e.g., liver cancer, lung cancer, bladder cancer, gastric cancer, breast cancer, uterine cancer, colon cancer, colorectal cancer, blood cancer, ovary cancer, and prostate cancer. Cancer screening may be carried out by the cancer gene analysis kit in a simpler and quicker manner with low costs.

Embodiments of the present disclosure are described below in greater detail.

Embodiments as disclosed herein are merely an example and are not intended to limit the scope of the present disclosure.

### <Embodiment> Amplification of Cancer Gene Variation

A. Extraction of DNA

DNA was extracted in order as follows.

300 [symbol] of blood was taken. A 3 volume of RBC buffer was added to the blood. The mixture was vortex-stirred and was then stayed in ice for 10 minutes. Thereafter, the mixture was centrifugated for one minute at 6.000 xg (>8,000 rpm). Then, the top layer was removed from the liquid, and white blood cells were then obtained the liquid.

300 [symbol] TG buffer and 20 mg/ [symbol] of proteinase K solution were added and mixed up with the precipitated cells, and the mixture were left to react with each other at 70° C.

After the reaction, 400 [symbol] of TB buffer (containing 80% ethanol) was added and mixed with the mixture, and the resultant mixture was put in a column, then centrifugated for one minute (at 6,000×g). The column was then put in a new tube.

After adding 500 [symbol] of washing buffer, centrifugation was performed for one minute. Then, the liquid collected in the to was discarded. Then, additional centrifugation was carried out on an empty tube for two minutes at the maximum speed, and the column was put in a new 1.5 [symbol] tube.

100 [symbol] of dilution buffer was put in the center of the column, left for two minutes at room temperature, then centrifugated for one minute at the maximum speed, and then the obtained DNA was stored at 4° C.

(B) Conversion of Cytosine of Thymine in DNA Base Sequence (DNA C/T Conversion)

40 [symbol] of 3-volume lysis buffer and proteinase K (500 ug/ml) were added to a cancer tissue and blood plasma sample and incubated at 65° C. for 30 minutes. Then, 6M NaCl 1.5 [symbol] and 4 [symbol] of chloroform were added to the culture liquid, and the mixture was centrifugated at 13,000 rpm for five minutes. Then, ethanol was added to the mixture, obtaining genomic DNA from the cancer tissue and blood plasma sample. The obtained DNA was dissolved in a TE buffer and was then stored. Here, as the buffer for dissolution, a mixed solution containing Tris-HCl (pH 8.0), 10 mM NaCl and 0.5% SDS was used. As the TE buffer, a mixed solution containing 10 mM Tris-HCl (pH 7.4) and 1 mM EDTA was used. For the DNA, two types, e.g., an Sssl methylase (CpG methylase. NEB, United Kingdom) treated group and a not-treated group, were used as a negative control group and a positive control group, respectively.

The concentration of the genomic DNA obtained from the cancer tissue and blood plasma sample was measured. Then, the A260/A280 ratio was compared to identify the purity of the DNA. In such manner, pure DNA which is average 3 ug in 200 [symbol] of blood could be obtained. The obtained DNA was treated with sodium bisulphite (NaHSO3), converting the CpG cytosine into thymine.

For reference, the treatment of DNA with NaHSO3 converts the non-methylated cytosine into thymine. However, cytosine that has been methylated remains cytosine with no change. Thus, different markings may be made depending on whether the cytosine has been methylated or not.

The DNA was treated with sodium bisulphite using the differences as to whether the DNA has been methylated and was analyzed by a methylation-specific primer PCR (MSP PCR) method, and whether the CpG island has been methylated was examined in the following order.

To examine whether the DNA has been methylated, the CT conversion reagent and M-dilution buffer were prepared from among the reagents contained in the EZ DNA methylation kit (Zymo Research).

The CT conversion reagent was prepared by adding 750 [symbol] of distilled water and 210 [symbol] cf M-dilution buffer to the C/T conversion reagent and mixing them for five minutes. The M-dilution buffer was prepared by adding 24 [symbol] of ethanol to the M-wash buffer contained in the EZ DNA methylation kit and mixing them. The CT conversion reagent and the M-dilution buffer prepared as such were used to perform the DNA methylation process using normal DNA and DNA extracted from a patient as set forth below.

5 ul M-dilution buffer was added to 1 ug of DNA, and distilled water was then added thereto so that the overall volume becomes 50 ul, thereby preparing an analysis sample. The prepared analysis sample was incubated at 37° C. for 15 minutes, and then, the reaction was terminated. 100 [symbol] of CT conversion reagent was added and mixed with each analysis sample whose reaction was terminated, and the analysis sample was sealed off with a foil and then incubated in an incubator at 50° C. for four hours to five hours.

The incubated sample was left in ice for 10 minutes. Then, 400 [symbol] of binding buffer (e.g., M-binding buffer) was added to the incubated sample and was carefully pipetted, thereby preparing a mixed sample. The mixed sample was injected into a Zymo-Spin I Column, to transferred to a 2 ml-collection tube, and then centrifugated at 13,000 rpm for 15 seconds to 30 seconds. Then, 200 [symbol] of M-wash buffer was added thereto and was centrifugated at 13,000 for 15 seconds to 30 seconds.

After the centrifugation, 200 [symbol] of M-desulphonation buffer was added to the mixture and was left at room temperature for 15 minutes. Then, the mixture was centrifugated at 13,000 rpm for 15 seconds to 30 seconds. 200 [symbol] of wash buffer was added to the mixture, and the resultant mixture was centrifugated at 13,000 rpm for 30 seconds. After the centrifugation, 10 [symbol] of dilution buffer was added to the mixture for dissolution, and was then centrifugated at 13,000 rpm for 30 seconds. Then, a DNA methylation process was performed thereon, thereby obtaining a nucleic acid sample containing the methylated cytosine.

### <Embodiment 2> Design and Preparation of Primers

Based on the coding sequences including the variation portions of multiple genes, forward and reward primers capable of serving as per-gene variation-specific primers were designed, and were selected and designed to have single nucleotide polymorphism (SNP) sequences to exhibit enough PCT specificity by modifying a conventional primer design method. In particular, I refers to deoxyinosine in the sequences of Tables 3 and 4 below.

According to an embodiment of the present disclosure, the nucleotide variation-specific primers was rendered to basically have the structure as represented in Formula 1 to be hybridized with the target sequences with very high specificity. In Formula 1, Ap refers to the specificity portion having a hybridization sequence continuously complementary to the 3′, Bq refers to the separation portion containing three or more universal bases, Cr refers to the variation-specific portion, which is the portion substantially joined to the target sequence, and p, q, and r refer to the number of nucleotides.

5′-Ap-Bq-Cr-3′ [Formula]

In the primer having the structure represented in Formula 1 above, two specificity portions are physically and functionally separated by the separation portions, and the hybridization specificity of the entire primer structure is double-adjusted by the variation specificity portion and the variation adjacent specificity portion.

Further, the base corresponding to the nucleotide variation or the base hybridized with the variation were rendered to be positioned at the middle of the variation specificity portion, so that the difference in the Tm value according to a mismatch is larger while no DNA synthesis occurs due to the Taq polymerase upon mismatching. The base sequences of each DLP primer (MU1001) designed in relation to the mutation of antioncogene are shown in Table 3 below. In Table 3, the bold letters refer to the

TABLE 3

| No. | Gene | 5′-Primer | 3′-Primer |
|---|---|---|---|
| 1 | APC | GAGGT III CCACACAGAACTAACCTC | GGTTT III TTATGAGAAAAGCAAACC |
| 2 | GSTP | CAGCA III CTCTGAGCACCTGCTG | GTCTC III GGTGTAGATGAGGGAGAC |
| 3 | GSTT | GCTTT III GAACTCCCTGAAAAGCTAAAGC | CCACC III GTTGGGCTCAAATATACGGTGG |
| 4 | GSTM | GAGAT III TTCCTTACTGGTCCTCACATCTC | TGCTG III TCACCGGATCATGGCCAGCA |
| 5 | CDH1 | TCTCA III GCTTGGGTGAAAGAGTGAGA | GGTCC III GGTGGGTTATGGGACC |
| 6 | MLH | CGTTA III CGGACAGCGATCTCTAACG | GTAGG III CGTGCTCACGTTCTTCCTAC |
| 7 | BRAF | CAATG III GAATATCTGGGCCTACATTG | AGAAA III CACTCCATCGAGATTTCT |
| 8 | DMNT3 | AACTG III GAAAACTCGGTTTCAGTT | GTGTT III CAGGTAAATCAGCTAACAC |
| 9 | ESR | CGTGG III GGTTTTCCCTGCCACG | CTCCT III CCGGAGTGTATGCAGGAG |
| 10 | XRCC1 | GGCTG III GGGCTCTCTTCTTCAGTC | GAGGG III CAGACCTCTCAACCCTC |
| 11 | EGFR | CAGGT III CATTCATGCGTCTTCACCTG | TGCAG III CGTTGGGCATGAGCTGTA |
| 12 | RB | CATCT III GGTCTCATAAGACTTCCTGAGATG | GAGTT III CAGGCTGGCTCAAACAC |
| 13 | BCL2 | TGGGA III GCTCCTTCATCGTCCCA | GGACT III GTCCGGTATTCGCAGAAGTC |
| 14 | MTHFR | GATCC III GAAGGTGTCTGCGGGATC | TCGAT III GCGTGATGATGAAATCGA |
| 15 | JAK2 | CTTTC III GTCATGCTGAAAGTAGGAGAAAG | TGAAA III TAGTCCACAGTGTTTTCAGTTTCA |
| 16 | ERBB2 | CGATG III CCTCTGACGTCCATCG | GATGC III TGCAGCAGTCTCCGCATC |
| 17 | KRAS1 | DCAGC III TGTGGTAGTTGGAGCTGH | GACTC III CCATAGGTACATCTTCAGAGTC |
| 18 | PTEN | TGTCC III GTAAAGCTGGAAAGGGATA | CCAGT III CAAGATCTGAAGCTCTACTGG |

Under the Taq DNA polymerase reaction, the DLP primers as per the embodiment annealed the target nucleic acid sequences, and in the DLP primers, the 5's were cut and the 3's were extended by a mold-dependent nucleic acid polymerase having 5′→3′ nuclease activity, so that fluorescent reporter molecules are stuck into the DLP primers. In such a manner, it was assessed whether signals enough to detect the target nucleic acid sequences could be generated. To assess the occurrence of a signal, the inventors adopted about 20 species of mutation genes as target molds, used synthetic oligonucleotides for the genes as molds for ease of experiment, and measured the signals using a three-step melting method.

As the fluorescent dye, FAM dT, HEX dT, Red 610 dT, Texas red dT, Cy5 dT, and TAMRA dT was used. The concentration of the primer was verified through the QC report of the manufacturer (Bioneer Corporation, South Korea). The primer was resuspended with triple deionized, sterilized distilled water to have the final concentration of about 100 pmole/ul and was frozen and stored at −70° C. in aliquots. The sequence of the cancer gene-related methylation gene variation DLP primer (or DLP probe, ME1001) used in the instant embodiment is shown in Table 4 below. In Table 4, the bold letters refer to the positions where fluorescent dye is joined for signal amplification.

TABLE 4

| | | Primer Sequence(5′-3′) | |
|---|---|---|---|
| No | Gene | Forward | Reverse |
| 1 | P16 | GCGATIIITTATTAGAGGGTGGGGCGGATCGC | TTACGIIIGACCCCGAACCGCGACCGTAA |
| 2 | RAR-b | CGAATIIITCGAGAACGCGAGCGATTCG | TCGTTIIIGACCAATCCAACCGAAACGA |
| 3 | MGMT | GACCGIIIGGGTATGCGTCGATTCGGTC | CGTTTIIIGCGAAAACGAAACCGAAACG |
| 4 | SIGMA | GACGCIIITGGTAGTTTTTATGAAAGGCGTC | CGTGGIIICCTCTAACCGCCCACCACG |
| 5 | DAPK | GACGTIIIGGATAGTCGGATCGAGTTAACGTC | TCGGCIIICCCTCCCAAACGCCGA |
| 6 | APC | GACCCIIITATTGCGGAGTGCGGGTC | TCGTCIIITCGACGAACTCCCGACGA |
| 7 | Ecad | ACGCGIIITTAGGTTAGAGGGTTATCGCGT | GTCGGIIITAACTAAAAATTCACCTACCGAC |
| 8 | hMLH1 | GCGACIIIACGTAGACGTTTTATTAGGGTCGC | CGCGGIIICCTCATCGTAACTACCCGCG |
| 9 | RASSF1A | GATACIIIGTGTTAACGCGTTGCGTATC | TCGTTIIIAACCCCGCGAACTAAAAACGA |
| 10 | CDH1 | GAAACIIIGTGGGCGGGTCGTTAGTTTC | CGTCGIIICTCACAAATACTTTACAATTCCGACG |
| 11 | HCAD | GCGAAIIITCGCGGGGTTCGTTTTTCGC | CGCGTIIIGACGTTTTCATTCATACACGCG |
| 12 | P15 | AACCGIIIGCGTTCGTATTTTGCGGTT | TCGGTIIIGGTACAATAACCGAACGACCGA |
| 13 | VHL | GCGTAIIITGGAGGATTTTTTTGCGTACGC | TTCGCIIIGAACCGAACGCCGCGAA |
| 14 | FHIT | TAAAAIIITTGGGGCGCGGGTTTGGGTTTTTA | TAGTGIIICGTAAACGACGCCGACCCCACTA |
| 15 | ESR1 | GACGAIIITTTTGGGATTGTATTTGTTTTCGTC | CGGGGIIIAACAAAATACAAACCGTATCCCCG |
| 16 | SRFP | GCGCGIIITGTAGTTTTCGGAGTTAGTGTCGCGC | CGTTCIIICCTACGATCGAAAACGACGCGAACG |
| 17 | RUNX3 | GAAACIIIGGCGGTTTTCGGTAGGTTTC | CGTCGIIIGAAACGAAACTAACGAAACGACG |
| 18 | BRCA | CGTTAIIICGGTAGTTTTTTGGTTTTCGTGGTAACG | CGATTIIITCAACGAACTCACGCCGCGCAATCG |

### <Embodiment 3> Assessment of Amplification Capacity of Multi-Amplification Primer for Detection of Nucleic Acid

The primer concentration and optimal conditions for PCR were established targeting 36 species of mutations and methylation positive standard substances for single nucleotide polymorphism (SNP) analysis of the target nucleic acids using the primers or primer set prepared by the method of embodiment 2.

To that end, 36 species of positive comparison substances and single nucleotide polymorphism (SNP) primers were used for amplification of the targets. The mixture of 10 pmole of mixed solution of 36 primers, 5 [symbol] of Hot start taq mastermix, a mixed solution of 50 species of positive standard substances, and negative standard substances was repeated 30 times for modification at 95° C. for 10 minutes, at 95° C. for 30 seconds, at 62° C. for 60 seconds, and at 72° C. for 60 seconds, and was then rendered to react at 72° C. for five minutes, thereby obtaining a reaction mixture. The tube containing the reaction mixture was first PCRed. The primer prepared as such was assessed for amplification capacity.

As a result of SNP analysis of 18 mutation genes, the amplification capacity of all the genes (APC, GSTP, GSTT, GSTM, CDH1, MLH1, BRAF, DMNT3B, ESR, XRCC1, EGFR, pRB, BCL2, MTHFR, JAK2, BRCA, ERBB2, PTEN) from the positive standard substances was verified, but the amplification capacity of all the single nucleotide polymorphism (SNP) primers was not verified from the negative standard substances. Based on such result, first MPCR conditions were established for second signal amplification.

The results of analysis of amplification capacity of the 18 methylation genes (P16, RAR-beta. MGMT, 14.3.3 sigma, DAPK, APC, Ecad, hMLH1, RASSF1A, CDH1, HCAD, P15, VHL, FHIT, ESR, RUNX3, BRCA, SRFP) revealed that amplification was verified for all the genes from the positive standard substances but not from the negative standard substances. Based on such results, first MPCR conditions were established for second signal amplification (refer to FIG. 2).

### <Embodiment 4> Analysis of Sensitivity of Product of Signal Amplification Using Product of Multiple Amplification

The assessment of amplification capacity relates to an analysis for second signal amplification using the product of the first amplification based on the conditions established in the first amplification capacity assessment, and its results are shown in FIG. 3.

To that end, 9 [symbol] of dilute product of first amplification, 1 [symbol] of each primer for MPCR, and 10 [symbol] of mastermix for melting analysis were mixed together. 10 [symbol] of the mixture was taken and was repeated 30 times for modification at 95° C. for 10 minutes, 95° C. for 20 seconds, 62° C. for 20 seconds, and 72° C. for 35 seconds. The signal generated during a predetermined time interval at each cycle was detected.

The product obtained by first amplifying 1 fg of positive comparison material in 1 ng was serial-diluted and was then MPCRed. As a result of second signal amplification using the product of the first amplification, the fluorescent signal of the target nucleic acid sequence could be verified up to 1 fg in the test for each positive substance concentration, but 1 fg revealed problems with reproductivity in the test. However, it could be verified that the minimum limit for detection was 10 fg, and the minimum limit for detection was accordingly set.

### <Embodiment 5> Specificity Analysis of Product of Signal Amplification Using Product of Multiple Amplification

Specificity analysis of the product of signal amplification using the product of multiple amplification was carried out to verify the amplification capacity for second signal amplification using the product of first amplification using the conditions established the first amplification capacity assessment, and the results were shown in FIGS. 4 and 5.

To that end, 9 [symbol] of diluted product of first amplification of embodiment 4, 1 [symbol] of mixed primer solution for MPCR analysis, and 10 [symbol] of mastermix for melting analysis were mixed together. 10 [symbol] of the mixture was taken and repeated 30 times for modifying at 95° C. for 10 minutes, 95° C. for 20 seconds, 62° C. for 20 seconds, and 72° C. for 35 seconds, and the signal was detected which was generated during a predetermined time interval at each cycle.

(1) When determined positive: for all positive DNA, positive dots other than those of a corresponding type were not shown even from the standard substance start concentration (ng/ml) at which a cross reaction is highly likely to occur, and the valid analysis value was less than 2.5×10̂1 (ng/ [symbol] ) the other types than the corresponding type.

(2) When determined negative: the negative standard substance showed no positive dots in the DLP TM Q Cancer detection kit, and the valid analysis value was 2.5×10̂1 ((ng/ [symbol] ) which was verified as not influencing the resultant value.

The homology percentage defined herein represents the portion that has homology relative to the overall gene size regardless of the length of primer. Thus, it could be verified that allele-specific PCR was determined according to the length of the overlapping portion separately from the entire length. From such results, the isoforms and transcript variations of the genes may sufficiently be analyzed if they have homology in some sequences and are determined to be able to affect the results of experiment. It can be known that the analyzed data supports the theoretical results. It is determined that when the items meet such references, the DLP TM Q Cancer detection kit may present performance fitting its unique purposes as well as reliable results.

While the present disclosure has been shown and described with reference to exemplary embodiments thereof, it will be apparent to those of ordinary skill in the art that various changes in form and detail may be made thereto without departing from the spirit and scope of the present disclosure as defined by the following claims.

What is claimed is:

1. A method for analyzing a cancer gene, the method comprising:
performing multiplex polymerase chain reaction (MPCR) on a nucleic acid sample extracted from a biological sample using a cancer gene-specific primer set including four or more species of sequences selected from the group consisting of sequence numbers 1 to 72;
performing multiplex real-time polymerase chain reaction (MRTPCR) on a product of the MPCR using the primer set and a marker; and
identifying the type of the cancer gene using a product of the MRTPCR, wherein the primer set includes an oligonucleotide of a dumbbell structure represented in Formula 1, wherein
5′-Ap-Bq-Cr-3′, [Formula 1]
wherein Ap is a specificity portion having a hybridization sequence continuous and complementary to the 3′, Bq is a separation portion including three or more universal bases, Cr is a variation specificity portion substantially joined to a target sequence, and p, q, and r each are the number of nucleotides.

2. The method of claim 1, wherein p and q each are an integer from 3 to 5, and r is an integer from 18 to 30.

3. The method of claim 1, wherein the MPCR includes preliminary modification at 95° C. for 10 minutes, repeating 30 times a series of steps including modification at 95° C. for 30 seconds, annealing at 62° C. for one minute, and expansion at 72° C. for one minute, and amplification at 72° C. for five minutes.

4. The method of claim 1, wherein the MRTPCR includes preliminary modification at 95° C. for 10 minutes and repeating 30 times a series of steps including modification at 95° C. for 20 seconds, annealing at 62° C. for 20 seconds, and expansion at 72° C. for 35 seconds.

5. The method of claim 1, wherein the marker includes one or more selected from the group consisting of FAM (5-carboxy fluorescein), ROX (carboxy-Xrhodamine), HEX (hexachlorofluorescein), Cal Fluor Red 610 (C46H57F6N5O4P2), Cy5 (cyanine-based fluorescent substance), Cy3, TAMRA (5-Carboxytetramethylrhodamine), Alexa 532, Rhodamine, tetra methyl rhodamine, Oregon green, R-PE, Alexa 546, Bodipy-TMRX, PBXL-1, Texas Red, Cryptofluor Orange, cyanine-based dye, and biotin

6. The method of claim 1, wherein identifying the type of the cancer gene includes heating the product of the MPCR at an interval of 4° C. to 5° C. per minute to 65° C. to 95° C. and analyzing a melting temperature curve according to a fluorescent signal.

7. A method for amplifying a cancer gene, the method comprising:
performing multiplex polymerase chain reaction (MPCR) on a nucleic acid sample extracted from a biological sample using a cancer gene-specific primer set including four or more species of sequences selected from the group consisting of sequence numbers 1 to 72;
screening the cancer gene by identifying a product of the MPCR, wherein the primer set includes an oligonucleotide of a dumbbell structure represented in Formula 1, wherein
5′-Ap-Bq-Cr-3′, [Formula 1]
wherein Ap is a specificity portion having a hybridization sequence continuous and complementary to the 3′, Bq is a separation portion including three or more universal bases, Cr is a variation specificity portion substantially joined to a target sequence, and p, q, and r each are the number of nucleotides.

8. A kit for analyzing a cancer gene, the kit comprising:
a primer set for analyzing the cancer gene, a dNTP mixture, a DNA polymerase, and a marker, wherein the primer set includes four or more species of sequences selected from the group consisting of sequence numbers 1 to 72, wherein the primer set includes an oligonucleotide of a dumbbell structure represented in Formula 1, wherein
5′-Ap-Bq-Cr-3′, [Formula 1]
wherein Ap is a specificity portion having a hybridization sequence continuous and complementary to the 3′, Bq is a separation portion including three or more universal bases, Cr is a variation specificity portion substantially joined to a target sequence, and p, q, and r each are the number of nucleotides.

9. The kit of claim 8, wherein the kit is used to simultaneously analyze gene mutation and methylation related to one or more species of cancer selected from the group consisting of liver cancer, lung cancer, bladder cancer, gastric cancer, breast cancer, uterine cancer, colon cancer, colorectal cancer, blood cancer, ovary cancer, and prostate cancer.
